(19) United States
(12) Patent Application Publication
(10) Pub. No.: US 2024/0241093 A1
(43) Pub. Date: Jul. 18, 2024

(54) TITER METHOD USING UV MEASUREMENT FOR CONTINUOUS BIOLOGICAL PRODUCTION

(71) Applicant: Boehringer Ingelheim International GmbH, Ingelheim am Rhein (DE)

(72) Inventors: Aaron KWONG, San Francisco, CA (US); Raquel OROZCO, Richmond, CA (US); Matthew Joseph STORK, Somerville, MA (US); Arch CREASY, Lawrence, MA (US); Zachary KYSER, Union City, CA (US)

(21) Appl. No.: 18/561,842

(22) PCT Filed: May 24, 2022
(86) PCT No.: PCT/US2022/030699

§ 371 Date: Nov. 17, 2023

Related U.S. Application Data
(60) Provisional application No. 63/192,642, filed on May 25, 2021.

(51) Int. Cl. G01N 30/86; C07K 1/22; G01N 21/59

(52) U.S. Cl. CPC G01N 30/8658; C07K 1/22; G01N 21/59

(57) ABSTRACT

Disclosed herein are methods of determining load volumes for capture chromatography in system and methods for continuous manufacture of a biological product.

### FIELD OF THE ART

Disclosed herein are methods for controlling the column loading of a protein for capture chromatography in a system and methods for continuous manufacture of a biologic product (e.g., a monoclonal antibody). Advantageously, the methods disclosed herein are automated and do not require an external device (e.g., HPLC).

### BACKGROUND

Perfusion cell culture is a highly efficient way to produce therapeutic proteins, where a perfusion membrane is used to continuously remove cell-free media as fresh media is added. The bioreactor permeate is then purified through a series of downstream unit operations. A common method of purification is multi-column bind-elute chromatography. In multi-column chromatography, two or more columns are loaded with the permeate stream. Once a column has reached its maximum loading capacity, loading switches to another column and the fully-loaded column is washed and eluted for further downstream operation. To establish how much volume of permeate can be loaded onto the column, the concentration of protein in the permeate must be determined. As an added complication, the concentration of protein in the permeate can change over time.

Frequent measurement of the concentration of protein becomes a key challenge then for a system consisting of a perfusion bioreactor linked directly to continuous capture chromatography. Measurements must be obtained once in real-time, e.g., every 4-12 up to 24 hours in order to capture any changes in titer that occur over time. Apart from the burden of measuring, it also creates the risk of contamination.

Various methods and products exist to determine protein concentrations and inform load volume for capture chromatography. The standard method to determine protein concentration in a complex feed is to use a high-performance liquid chromatography (HPLC)-based titer assay. Protein A HPLC is frequently used to determine the concentration of monoclonal antibody (mAb) in material with high levels of protein impurities. This method has been shown to be accurate and precise (Fernández et al. Development and Validation of an Affinity Chromatography-Protein G Method for IgG Quantification. Int Sch Res Notices. (2014) 2014:487101) and is currently the standard method for determining mAb titer for batch-based production processes. However, there are difficulties in using HPLC for measurement of titer in a continuous process, as running HPLC requires that a sample be taken from the process.

One method is to manually take samples from an aseptic sampling manifold, and then manually run the sample on an HPLC. This approach is labor intensive, as it requires an operator that can take frequent samples and run the HPLC.

Another option is to use an automated sampling device with an HPLC designed for in-line operation, such as the Waters Patrol. However, this requires that the HPLC instrument be placed in the GMP suite close to the sampling point, which is difficult from a compliance perspective. Additionally, HPLCs are relatively complex instruments with many moving parts, and instrument maintenance becomes more difficult if the instrument is placed in a GMP space.

More recently, spectroscopic methods have gained favor as a method for determination of protein concentration. International Publication No. WO2010151214A1 describes a spectroscopic method in which UV sensors were placed in the feed and effluent streams of a Protein A capture chromatography system.

There remains a need for methods of determining protein concentration and informing target load volumes in capture chromatography where the feedstream protein concentration varies over time.

### BRIEF SUMMARY OF THE INVENTION

The subject technology is illustrated, for example, according to various aspects and embodiments listed below.

In one aspect a method for optimizing continuous chromatography (e.g., continuous protein A chromatography) in a system of continuous production of a biologic product (e.g. a perfusion bioreactor) is disclosed, comprising (i) taking an optical density (OD) measurement of the eluate from the chromatography column; (ii) applying a mathematical formula to the OD measurement, wherein the mathematical formula is Equation 1; and (iii) adjusting the continuous load of the chromatography column to achieve the optimization.

$$Titer = \frac{{k}_{c o l}}{2 {ε}_{m} {V}_{l}} ( {\sum }_{i = 0}^{n} {Q}_{i} {D}_{i} + {\sum }_{i = 0}^{n} {Q}_{i + 1} {D}_{i + 1} ) \qquad Equation 1$$

In another aspect, a method for optimizing continuous chromatography (e.g., continuous protein A chromatography), where the method is optionally computer implemented, in a system of continuous production of a biologic product (e.g. a perfusion bioreactor) is disclosed, comprising (i) taking an optical density (OD) measurement of the eluate from the chromatography column; (ii) applying a mathematical formula to the OD measurement, wherein the mathematical formula is Equation 2; and (iii) adjusting the continuous load of the chromatography column to achieve the optimization.

$$m = \frac{n \sum {x}_{1} {y}_{i} - ( \sum {x}_{i} ) ( \sum {y}_{i} )}{n \sum {x}_{i}^{2} - {( \sum {x}_{i} )}^{2}} \qquad Equation 2$$

$$b = \frac{\sum {y}_{i} - m \sum {x}_{i}}{n}$$

In certain embodiments, the methods disclosed herein permit an improvement in one or more properties selected from increased productivity, reduced facility footprint, more agile manufacturing processes and reduced overall specific cost-of-goods (COG).

In another aspect, a method of manufacturing a biologic product of interest is provided, the method comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) capturing or purifying the biologic product of interest comprising continuous chromatography of the fluid feed comprising biologic product of interest and one or more impurities or buffer components; and
(IV) optionally formulating the biologic product of interest into a pharmaceutically acceptable formulation suitable for administration; and
wherein the method further comprises:
(i) taking an optical density (OD) measurement of an eluate from a chromatography column; (ii) applying a mathematical formula to the OD measurement, wherein the mathematical formula is Equation 1 or Equation 2; and (iii) adjusting the continuous load of the chromatography column to achieve the optimization.

The term “adjusting” as used herein, refers to changing or updating the titer value used in DeltaV to determine the load volume requirement,

Additional advantages of the subject technology will become readily apparent to those skilled in this art from the following drawings and the detailed description. The drawings and description are to be regarded as illustrative in nature, and not as restrictive.

### BRIEF DESCRIPTION OF FIGURES

FIG. 1 depicts a continuous protein manufacturing system, for example, an iSkid system, as an exemplary embodiment of a continuous production system for which the disclosed method can be utilized.

FIGS. 2A and 2B show chromatograms from a representative Protein A chromatography cycle. The shaded area indicates the area under the curve for the elution peak, which is used to calculate the mass of product of product eluted from the column. Volume=0 is pegged to the start of collection of the eluate. FIG. 2A shows a chromatogram for a full chromatography cycle. FIG. 2B shows a zoomed view of the elution peak. The elution collection volume was 3L as indicated by the arrows.

FIG. 3 shows predicted titer using the “Feed Minus Effluent” Difference Method. Absorbance was measured at 300 nm. Volume was normalized. Eight separate continuous batches across three different sites have been included. Inlet UV data was not available for all cycles, so some cycles have been excluded from this dataset.

FIG. 4 shows predicted vs. actual titer using the elution UV titer prediction method. Titer predictions were compared against the actual bioreactor permeate protein concentration at the time of elution.

FIG. 5 shows predicted vs. actual titer when employing the linear regression adjustment method to the titer predictions.

FIGS. 6A, 6B, 6C and 6D show expected load challenges for the Protein A column based on the elution UV titer prediction method. FIG. 6A shows the full dataset. FIG. 6B shows the dataset with runs removed where column overloading (>65 g/L challenge) led to underprediction. FIG. 6C shows the full dataset with the linear regression correction. FIG. 6D shows the linear regression correction method with overloaded runs removed.

FIG. 7 depicts the decision tree for the, optionally, computer-implemented method disclosed herein.

### DETAILED DESCRIPTION OF THE INVENTION

As the commercial relevance of continuous biomanufacturing grows, the processing of large quantities of biomolecules (e.g., proteins, antibodies) from crude solutions (e.g., cell culture media) has become increasingly important.

Typically, purification is a multi-step process. One important step within the multi-step process is chromatography, which separates the biologic product of interest from one or more components, e.g., contaminants. Various strategies for using chromatography to purify a molecule of interest are known in the art as well as various modes of operating the same.

Disclosed herein is a method and system for continuous downstream processing which permits optimized, continuous loading of chromatography unit operation (e.g., protein A columns) to permit capture/purification of the biologic product. In particular, the systems and methods herein address the relatively high and dynamic titers associated with a perfusion cell culture process. A dynamic titer is one in which concentration of protein varies over time, i.e., is non-uniform. In certain embodiments, the concentration may differ by about 5%, about 10%, about 20%, about 25% or more over time.

In certain embodiments, the methods and systems disclosed herein process up to 6 g/L/day (g of protein/L of bioreactor volume/day). In one embodiment, the methods and systems disclosed herein process about 3 g/L/day, about 4 g/L/day, about 5 g/L/day or about 6 g/L/day or more.

Specifically, the systems and methods herein permit the load density to remain within a predetermined, target load density range and prevent both underloading (e.g., less than about 20 g/L-resin) and overloading (>90% of DBC at 10% breakthrough, e.g., about 65 g/L or more) of the chromatography column. In some embodiments, the method permits utilization of existing equipment, i.e., does not require an existing production system to be modified and/or incorporate equipment external to an existing production system.

The disclosed methods and systems permit one or more improvements selected from increased productivity, reduced facility footprint, agile manufacturing processes and reduced overall specific cost-of-goods (COG). The disclosed methods and systems also reduce the need for human intervention in processing of the biologic product, which otherwise contributes to cost, error and contamination. In particular, the methods and systems disclosed herein permit optimized loading densities and loading volumes with respect to the sample (e.g., feed stream) loaded onto a chromatography column. In certain embodiments, the methods and systems disclosed herein determine load quantity, then calculate the volume required to achieve the load quantity.

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

To facilitate an understanding of the present subject technology, a number of terms and phrases are defined below:

### I. Definitions:

The grammatical articles “one”, “a”, “an”, and “the”, as used herein, are intended to include “at least one” or “one or more”, unless otherwise indicated. Thus, the articles are used herein to refer to one or more than one (i.e., to at least one) of the grammatical objects of the article. By way of example, “a component” means one or more components, and thus, possibly, more than one component is contemplated and may be employed or used in an implementation of the described embodiments.

The term “about” generally refers to a slight error in a measurement, often stated as a range of values that contain the true value within a certain confidence level (usually ±1σ for 68% C.I.). The term “about” may also be described as an integer and values of ±20% of the integer.

The term “affinity” refers to the strength of binding of a single molecule (e.g., a protein) to its ligand. It is typically measured and reported by the equilibrium dissociation constant (KD), which is used to evaluate and rank order strengths of biomolecular interactions.

The terms “affinity chromatography” and “protein affinity chromatography,” as used interchangeably herein, refer to a separation technique in which a target biologic product (e.g., an Fc region containing protein of interest or antibody) is specifically bound to a ligand which is specific for the target biologic product, i.e. an affinity ligand. In some embodiments, the ligand (e.g., protein A or a functional variant thereof) is covalently attached to a chromatographic solid phase material and is accessible to the target protein in solution as the solution contacts the chromatographic solid phase material. The target biologic product generally retains its specific binding affinity for the ligand during the chromatographic steps, while other solutes and/or proteins in the mixture do not bind appreciably or specifically to the ligand. Binding of the target to the immobilized ligand allows contaminating proteins or protein impurities to be passed through the chromatographic medium while the target biologic product remains specifically bound to the immobilized ligand on the solid phase material. The specifically bound target biologic product is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column. Any component can be used as a ligand for purifying its respective specific binding protein, e.g. antibody. However, in various methods according to the present invention, protein A is used as a ligand for an Fc region containing target protein. The conditions for elution from the ligand (e.g., protein A) of the target biologic product (e.g., an Fc region containing protein) can be readily determined by one of ordinary skill in the art. In some embodiments, the ligand is not protein A.

The term “affinity ligand” as used herein refers to a molecule which has specific non-covalent binding capability to other molecules.

The term “antibody” and “immunoglobulin” are used interchangeably herein, and are understood to include also fragments of antibodies, fusion proteins comprising antibodies or antibody fragments and conjugates comprising antibodies or antibody fragments. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The antibody may be, for example, a polyclonal antibody, a monoclonal antibody, a human antibody, a humanized antibody, a chimeric antibody, a bi-specific antibody or a multi-specific antibody.

The term “area under the curve” or “AUC” refers to the area between the x-axis and the curve given by the integrand. It is equal to the definite integral of a function.

The term “bioreactor” as used herein refers to an apparatus in which a biological reaction or process is carried out. Current bioreactor system options are batch, fed-batch, and continuous (i.e., perfusion). Examples of bioreactors include culture bioreactors and production bioreactors.

The term “binding” as used herein refers to a step during which resin and unpurified target biologic product form a reversible complex (for positive chromatography), or during which resin and impurities form a reversible complex (for negative chromatography).

The term “binding fragment” as used herein refers to a Fab, a Fab′, a F(ab′)2, a scFv, a scFab, a dsFv, a ds-scFv, dimers (e.g. Fc dimers), minibodies, diabodies and multimers thereof, multispecific antibody fragments and domain antibodies.

The term “biologic product” as used herein generally refers to a product of interest created via biological processes or via the chemical or catalytic modification of an existing biologic product. Biological processes include cell culture, fermentation, metabolization, respiration, and the like. Biologic products of interest include, for example, antibodies, antibody fragments, proteins, hormones, vaccines, fragments of natural proteins (such as fragments of bacterial toxins used as vaccines, e.g., tetanus toxoid), fusion proteins or peptide conjugates (e.g., such as subunit vaccines), virus-like particles (VLPs) and the like. In certain embodiments, the biologic product has UV-Vis absorbance in the range of 190 to 700 nm.

The term “buffer” as used herein refers a solution that resists changes in pH by the action of its acid-base conjugate components.

The term “capturing” as used herein refers to a step performed to partially purify or isolate (e.g., at least or about 5%, e.g. at least or about 10%, 15%, 20%, 25%, 30%, 40%, 45%, 50%, 55%, 60%, 65%, 70%, 75%, 80%, 85%, 90%, or at least or about 95% pure by weight), concentrate, and stabilize a protein of interest (e.g., a recombinant therapeutic protein) from one or more other components present in a liquid culture medium or a diluted liquid culture medium (e.g., culture medium proteins or one or more other components (e.g., DNA, RNA, or other proteins) present in or secreted from a mammalian cell). Typically, capturing is performed using a resin that binds a protein of interest (e.g., through the use of affinity chromatography).

The term “cell culture” as used herein refers to cells in a liquid medium. Optionally, the cell culture is contained in a bioreactor. The cells in a cell culture can be from any organism including, for example, bacteria, fungus, insects, mammals or plants. In a particular embodiment, the cells in a cell culture include cells transfected with an expression construct containing a nucleic acid that encodes a protein of interest (e.g., an antibody).

The term “cell culture medium” as used herein refers any type of media used in the context of culturing cells. Typically, a cell culture medium comprises amino acids, at least one carbohydrate as an energy source, trace elements, vitamins, salts and possibly additional components (e.g. in order to influence cell growth and/or productivity and/or product quality).

The term “chromatography” as used herein generally refers to a group of techniques for the separation of mixtures. The mixture is dissolved in a fluid called the mobile phase, which carries it through a structure holding another material called the stationary phase. The stationary phase may be referred to as a “resin”. The various constituents of the mixture travel at different speeds, causing them to separate. The separation is based on differential partitioning between the mobile and stationary phases. Subtle differences in a compound's partition coefficient result in differential retention on the stationary phase and thus changing the separation. In column chromatography, the mobile phase, or eluent, is pumped through the column filled with a stationary chromatography resin, wherein the components to be separated travel through the column at different speeds and are collected at different times at the outlet of the column. Molecules that are more attracted to the stationary phase move more slowly through the system than those that are more attracted to the mobile phase. Since the eluent is pumped through the column at a defined flow rate, this also means that molecules will elute after different volumes of eluent have passed through the column. This is captured in a chromatogram, which is a plot of the concentration exiting the column versus time or volume. The retention volume, VR, for a molecule is the volume that has passed through the column since the target molecule was introduced onto the column.

The terms “chromatography resin” or “chromatography media” are used interchangeably herein and refer to any kind of solid phase which separates an analyte of interest (e.g., an Fc region containing protein such as an immunoglobulin) from other molecules present in a mixture. Usually, the analyte of interest is separated from other molecules as a result of differences in rates at which the individual molecules of the mixture migrate through a stationary solid phase under the influence of a moving phase, or in bind and elute processes. Non-limiting examples include cation exchange resins, affinity resins, anion exchange resins, anion exchange membranes, hydrophobic interaction resins and ion exchange monoliths.

The term “contaminants” as used herein refers to any undesired component or compound within a mixture. In cell cultures, cell lysates, or clarified bulk (e.g., clarified cell culture supernatant), contaminants include, for example, host cell nucleic acids (e.g., DNA) and host cell proteins present in a cell culture medium. Host cell contaminant proteins include, without limitation, those naturally or recombinantly produced by the host cell, as well as proteins related to or derived from the protein of interest (e.g., proteolytic fragments) and other process related contaminants. In certain embodiments, the contaminant precipitate is separated from the cell culture using an art-recognized means, such as centrifugation, sterile filtration, depth filtration and tangential flow filtration.

The term “continuous” as used herein has different meaning with respect to a unit operation and a process. A unit operation is continuous if it is capable of processing a continuous flow input for prolonged periods of time. A continuous unit operation has minimal internal hold volume. The output can be continuous or discretized in small packets produced in a cyclic manner. A process is continuous if it is composed of integrated (physically connected) continuous unit operations with zero or minimal hold volume in between and suitable controls are in place to capture process variation. See Konstantinov et al., Journal of Pharmaceutical Sciences 104, no. 3 (March 2015): 813-20. Ideally, a continuous process is regulated so that, to the greatest extent possible, every step or unit operation of the continuous process is running at the same time and at substantially the same production rate. In this way, compression of the cycle time is maximized and the shortest possible completion time is achieved.

The term “column” as used herein refers to a vessel, including, for example, one or more tubes, within which separation of compounds occurs.

The term “column saturation” as used herein refers to the point where the column is close 100% of its dynamic binding capacity and loading additional product at the inlet would not result in more capacity.

The term “cycle” as used herein refers to a multi-step process which starts with equilibration of the chromatography column with a neutral buffer; followed by loading of a clarified feed stream to the column, where the clarified feed stream contains the biologic product (e.g., antibody); followed by washing the column to remove loosely bound impurities, followed by eluting the target biologic molecule off of the column. This multi-step process of equilibration, loading, washing and elution constitutes a cycle or a bind and elute cycle.

The term “downstream” or “downstream processing” as used herein generally refers to some or all the steps necessary for capture of a biologic product from the original solution in which it was created, for purification of the biologic product away from undesired components and impurities, for filtration or deactivation of pathogens (e.g. viruses, endotoxins), and for formulation and packaging.

The term “dynamic binding capacity” or “DBC” as used herein refers to the available capacity of a stationary phase as a function of loading flow velocity.

The term “eluate/filtrate” as used herein refers to a fluid that is emitted from a chromatography column or chromatographic membrane that contains a detectable amount of a target biologic product (e.g., monoclonal antibody).

The term “elution” as used herein refers to a step in which the complex of resin and the target biologic product is reversed and the purified product is collected.

The term “feed stream” as used herein refers to refers to the raw material or raw solution derived from a production (upstream) scheme that is delivered to the initial unit operation, which raw material contains the biologic product of interest (e.g. protein, polypeptide, antibody, etc.) and may further contain various contaminants (e.g., non-desired proteins, cell fragments, viruses, DNA).

The terms “holding tank,” “pool tank,” and “intermediate tank,” as used interchangeably herein, refer to any container, tank or bag, which may be used to collect the output of a process step (e.g., an eluate from a column). In most conventional processes, one or more intermediate containers are used for adjusting the conditions/properties of the output from one process step to make it suitable for the next process step. For example, it may be necessary to adjust the pH and/or conductivity of an eluate from a chromatography column or step (e.g., containing protein target and impurities) before loading the pool onto the next chromatography column or step. In various embodiments according to the methods of the present invention, the need for a holding tank is obviated.

The term “immunoglobulin binding domain” as used herein refers to a domain that can bind to a constant region of immunoglobulins (e.g., Fc of IgG).

The term “KD” as used herein refers to the equilibrium dissociation constant, a ratio of koff/kon, between the affinity ligand and a molecule. The lower the KD value, the higher the affinity.

The term “integrated” as used herein with respect to a process refers to a process which is performed using structural elements that function cooperatively to achieve a specific result (e.g., the generation of a therapeutic protein drug substance from a liquid culture medium).

The term “isolated biologic product” as used herein refers a product substantially free of cellular material or culture medium when produced by recombinant DNA techniques.

The term “load density” or “load challenge” as used herein refers to the total mass of product loaded onto the column in the load cycle of a chromatography step or applied to the resin in batch binding, measured in units of mass of product per unit volume of resin.

The term “load time” as used herein refers to the amount of time required to load a column in the load phase of a chromatography purification process.

The term “load volume” refers to the volume of liquid sample (e.g., feed stream) that is loaded onto a chromatography column.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method.

The term “multi-column chromatography” or “MCC” as used herein refers to a two or more interconnected or switching chromatography columns and/or chromatographic membranes in sequential order in series or in parallel. In certain embodiments, herein the multi-column chromatography system includes two chromatography columns in sequential order in series.

The term “non-uniform” as used herein refers to a varied or changing quality or appearance. In one embodiment herein, the column is loaded with a non-uniform stream.

The term “overload” as used herein with reference to a mode of chromatography in which the target biologic product (e.g., monoclonal antibody) is loaded beyond the DBC of the chromatography material for the product, thus referred to as overload. The methods and systems disclosed herein do not achieve overloading.

The term “perfusion cell culture” as used herein refers to perfusion cultivation which is carried out by continuously feeding fresh medium to the bioreactor and constantly removing the cell-free spent medium while retaining the cells in the reactor; thus, a higher cell density can be obtained in perfusion cultures compared to continuous cultures, as cells are retained within the reactor via a cell retention device. The perfusion rate depends on the demands of the cell line, the concentration of nutrients in the feed and the level of toxification.

The terms “polypeptide”, “polypeptide product”, “protein” and “protein product,” are used interchangeably herein to refer to a molecule consisting of two or more amino acids, e.g., at least one chain of amino acids linked via sequential peptide bonds. In one embodiment, a “protein of interest” or a “polypeptide of interest” is a protein encoded by an exogenous nucleic acid molecule that has been transformed into a host cell, wherein the exogenous DNA determines the sequence of amino acids. In another embodiment, a “protein of interest” is a protein encoded by a nucleic acid molecule that is endogenous to the host cell.

The term “protein A” and “ProA” are used interchangeably herein and encompasses protein A recovered from a native source thereof, protein A produced synthetically (e.g., by peptide synthesis or by recombinant techniques), and variants thereof which retain the ability to bind proteins which have a CH₂/CH₃ region, such as an Fc region. Protein A can be purchased commercially from Repligen, Pharmacia and Fermatech. Protein A is generally immobilized on a solid phase support material. The term “ProA” also refers to an affinity chromatography resin or column containing chromatographic solid support matrix to which is covalently attached protein A.

The terms “purifying” as used herein refers to a step performed to isolate a biologic product of interest (e.g., a monoclonal antibody) from one or more other impurities (e.g., bulk impurities) or components present in a fluid containing a protein of interest (e.g., liquid culture medium proteins or one or more other components, e.g. DNA, RNA, other proteins, endotoxins, viruses, etc.) present in or secreted from a mammalian cell). For example, purifying can be performed during or after an initial capturing step. Purification can be performed using a resin, membrane, or any other solid support that binds either the biologic product of interest or contaminants (e.g. through the use of affinity chromatography, hydrophobic interaction chromatography, anion or cation exchange chromatography, or molecular sieve chromatography). A protein of interest can be purified from a fluid containing the protein using at least one chromatography column and/or chromatographic membrane (e.g., any of the chromatography columns or chromatographic membranes described herein).

The term “regeneration” as used herein with reference to a step or mode indicates an operation during which the resin is cleaned for the purpose of reuse or for later cycles.

The term “retention time” refers to the time in which half of the quantity of a solute is eluted from the chromatographic system. It is determined by the length of the column and the migration velocity of the solute.

The term “semi-continuous,” in the context of liquid transfer to and/or from a bioreactor, as used herein means ‘periodic’ or refers to a scenario in which liquid (e.g. media alone and/or with cells, cell bleed) is added to and/or removed from the bioreactor once every however long period of time. For example, once every 1, 2, 5, 10, 15, 30, 45 or 60 minutes, or once every hour, or once every 2-3 hours, or once every however long period of time from 1 minutes to 24 hours, a burst of liquid is transferred from and/or to the bioreactor for a period extending from few seconds (e.g. 1 sec., 2 sec., 5 sec. 10 sec., 20 sec. or 60 sec.) to several minutes (e.g. 2 min. 5 min., 10 min., 25 min., 50 min, 120 min. or 240 min.).

The term “sequential” as used herein with respect to chromatography refers to chromatography steps in a specific sequence; e.g., a first chromatography step followed by a second chromatography step followed by a third chromatography step, etc. Additional steps may be included between the sequential chromatography steps.

The term “titer” as used herein refers to the total amount of protein produced by a cell culture, divided by a given amount of medium volume. In essence, the term “titer” refers to a concentration and is typically expressed in units of milligrams of polypeptide per liter of medium. The methods of the present invention have the effect of substantially increasing polypeptide product titer, as compared to polypeptide product titer produced from other cell culture methods known in the art.

The term “upstream” or “upstream process” as used herein generally refers to the step(s) of biopharmaceutical manufacture relating to the creation of the active biologic product by a biological process or other reaction. Ordinarily, the biologic product to be isolated and processed into a biopharmaceutical is the result of a fermentation or is the expression product of a recombinantly transformed host cell. Upstream processes involving creation of a biologic product in cell culture will be conducted in a fermenter or bioreactor, and the upstream process may be a batch process (e.g., batch or fed-batch cell culture grown in a fermenter) or a continuous process (e.g. perfusion cell culture).

The term “washing” as used herein refers to a step in which resin with bound product is washed with a washing buffer to rid the resin of impurities (for positive chromatography), or during which resin with bound impurities is washed with a washing buffer to wash out carryover product from the binding step (for negative chromatography).

### II. Continuous Biological Production System

In one embodiment, the methods disclosed herein are useful in connections with methods and systems for continuous production of a biologic product (e.g., a monoclonal antibody).

In certain embodiment, the system is an integrated, continuous biological production system for production of a biologic product that includes continuous upstream and downstream processes and more particularly, a perfusion bioreactor coupled to a continuous capture (purification) function. In other embodiments, the integrated, continuous biological production system further includes one or more additional downstream processes selected from viral inactivation, filtration, formulation, filing and combination thereof.

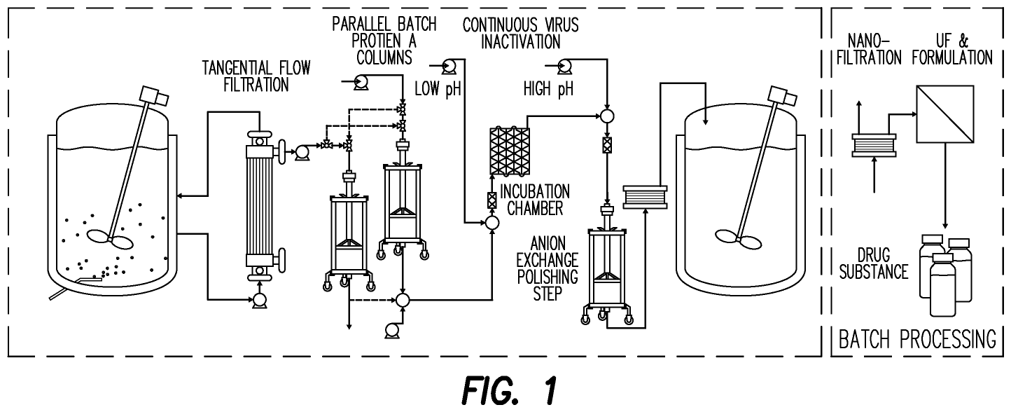

In one embodiment, the system is a continuous protein manufacturing system, for example, an iSKID system, a fully integrated and automated system that hydraulically links the perfusion bioreactor with several downstream unit operations (2× Protein A columns, continuous viral inactivation, anion-exchange chromatography in flow through mode, and single-pass tangential flow filtration (SPTFF). See FIG. 1.

Within the system, capture or purification of the target biologic product (e.g., monoclonal antibody) may be achieved utilizing continuous chromatography.

In certain embodiments, purification is provided by one column continuous chromatography consisting of a single column integrated with continuous upstream processes. The approach applies a combination of perfusion rate and loading flow-rate to control the column loading and non-loading steps. The complexity of the process control is reduced as compared to the multi-column operations wherein only one column needs to be monitored.

In one embodiment, purification is provided by multi-column continuous chromatography to create a load zone in lieu of one industrial scale column. Multi-column chromatography generally require smaller column volumes and reduced buffer volumes compared to a standard batch process, while producing the same amount of biologic. Examples of multi-column approaches include sequential multi-column chromatography, (SMCC), three column periodic chromatography (3C-PCC) and two column chromatography capture SMB (2C-PCC).

In one embodiment, purification is provided by at least two sequential chromatography columns. During operation, one of the two columns is always loading, and at the same time, the other column is processing. The basic sequence is wash, elute, regenerate, equilibrate and wait for the next load.

The size of the chromatography column may vary. In one embodiment, the chromatography column has a volume of about 1 mL, about 2 mL, about 3 mL, about 4 mL, about 5 mL, about 6 mL, about 7 mL, about 8 mL, about 9 mL, about 10 mL, about 15 mL, about 20 mL, about 25 mL, about 30 mL, about 40 mL, about 50 mL, about 75 mL, about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, about 10 L, about 25 L, about 50 L, about 100 L, about 200 L, 300 L, about 400 L, about 500 L, about 600 L, about 700 L, about 800 L, about 900 L or about 1000 L or more.

The type of chromatography column may vary. In certain embodiments, the chromatography resin is an affinity chromatography resin including an affinity ligand. Any affinity ligand may be used in the systems and method provided that the ligand is a specific binding partner of a target biologic product of interest.

In one embodiment, the affinity ligand has an immunoglobulin domain, such as an Fc-binding domain. The affinity ligand may be a full-length protein or a functional variant of a full-length protein. The affinity ligand may be a monomer, dimer or multimer of a full-length protein or functional variant.

In one embodiment, the affinity ligand may be protein A, protein G or functional variants of either, where the target molecule could be an immunoglobulin or Fc region of an immunoglobulin or a molecule comprised of at least a portion of an Fc region of an immunoglobulin. In certain embodiments, the affinity ligand is a bacterial immunoglobulin binding protein.

In some embodiments, the affinity ligand is protein A or a component thereof. Protein A can be a native (e.g. Staphyloccocus aureus) or a recombinant protein A coupled with a natural (agarose or cellulose) or synthetic (polyvinylether, polystyrene-divinyl benzene, pore glass, or polymethacrylate) base matrix.

In a particular embodiment, the affinity ligand is selected from the B, C, A, E, D domains derived from Staphylococcus protein A or a functional variant thereof. Each of the E, D, A, B and C domains possess distinct immunoglobulin binding sites. One site is for Fc (the constant region of IgG class of g) and the other is for the Fab portion of certain Ig molecules (the portion of the Ig that is responsible for antigen recognition). It has been reported that each of the domains contains a Fab binding site. The non-immunoglobulin binding portion is located at the C-terminus and is designated the X region or X-domain.

Exemplary protein A based resins which may be used in the methods of the invention include, but are not limited, to PROSEP vA High Capacity, PROSEP A Ultra, PROSEP Ultra Plus (Millipore), Protein A Sepharose FastFlow, rmp Protein A Sepharose FastFlow, MabSelect, MabSelect Xtra, MabSelect SuRe (GE Healthcare), POROS A, POROS MabCapture A (Applied Biosystems), and Sartobind Protein A (Sartorius).

In one embodiment, the affinity ligand is protein G. Protein G is an immunoglobulin-binding protein expressed in Streptococcal bacteria. It is similar to protein A but it has differing binding specificities. In a particular embodiment, the affinity ligand is a B domain derived from Staphylococcus protein B or a functional variant thereof.

Exemplary protein G based resins include, but are not limited, to PROSEP-G (Millipore), Protein G Sepharose™. 4 Fast Flow (GE Healthcare), POROS G (Applied Biosystems).

In certain embodiments, the affinity ligand is not a bacterial immunoglobulin binding protein but rather an alternative affinity ligand such as a synthetic binding protein, peptide, aptamer or synthetic small molecule compound.

The KD of the affinity ligand vary. In one embodiment, the affinity ligand has a KD of between about 1 nM to 1 uM, and more particularly, about 10 nM.

Capturing the target biologic product may comprise (i) contacting a chromatography matrix (e.g. comprised of an affinity chromatography media with a mixture comprising a target biologic product under conditions such that the target molecule preferentially binds to the chromatography matrix and (ii) optionally eluting the target biologic product from the matrix by altering one or more conditions, e.g. by applying an elution buffer. In one embodiment, the elution is a step elution or gradient elution. The method may optionally include one or more wash steps. The wash steps may be performed for example after the target molecule has bound to the matrix, hut before the target molecule has been eluted from the matrix. Additional wash steps may optionally be performed after the elation of the target molecule, e.g., to clean the matrix of residually bound material.

Any suitable buffer may be employed. Selection of the buffer may depend, for example, on the desired pH, the characteristics of the biologic product of interest (e.g., monoclonal antibody), the chromatography material and other variables familiar to those of ordinary skill. See A Guide for the Preparation and Use of Buffers in Biological Systems. Gueffroy, D., Ed. Calbiochem Corporation (1975).

The elution buffer may have for example a pH which differs from the pH of the mixture as it was applied to the matrix, or it may have a higher concentration of salt as compared to the original mixture applied to the matrix

In certain embodiments, the capture or purification process consists of loading an affinity chromatography column with a fluid sample (e.g., cell culture medium or clarified cell culture medium) including the target biologic product (e.g., monoclonal antibody), washing the column to remove unwanted biological material (e.g., contaminating proteins and/or small molecules), eluting the target biologic product bound to the column, and re-equilibrating the column.

In one embodiment, the capture or purification processes involves continuously feeding the fluid (e.g., liquid culture medium) into a first affinity chromatography column, capturing the target biologic product (e.g., monoclonal antibody) from the liquid, producing an eluate from the first affinity chromatography column that includes the target biologic product (e.g., monoclonal antibody) and continuously feeding the eluate into a second affinity chromatography column and subsequently eluting the target biologic product to produce a purified target biologic product.

The cell culture medium can be obtained from a perfusion cell (e.g., mammalian cell) culture (e.g., a perfusion bioreactor including a culture of mammalian cells that secrete the recombinant protein). Liquid cell culture medium can be filtered or clarified to obtain a liquid culture medium that is substantially free of cells and/or viruses.

The liquid cell culture medium can be continuously fed onto affinity chromatography column using a variety of different means, e.g., actively pumped into the first affinity chromatography column or fed into the same using gravitational force. The “loading” step involves loading the column by passing the fluid (the feed) through an inlet, such that the feed contacts the sorbent, and some amount of the target product is bound. Continuous loading permits integration with continuous upstream process.

To establish how much volume of the cell culture medium can be loaded onto the column, the concentration of the target biologic product (e.g., monoclonal antibody) in the cell culture medium must be determined. Batch production yields a single homogenous permeate, thus only one titer measurement (along with volume loaded) is sufficient to determine the mass loaded. During continuous production, however, permeate continuously exits a perfusion reactor and loads a chromatography column, so the product titer can vary over the loading period.

Generally, a user can determine when a chromatography column is finished loading by one of two parameters: time or load density. With time, the system stops loading after the time length given by the operator is reached. With load density, the volume totalizer from flow meters is utilized along with a “user”-derived titer average value to calculate the total mass of protein being loaded. Methods currently known in the art for utilizing load density reviewed above and generally rely on HPLC. In contrast, the methods disclosed herein (as discussed in more detailed below) involve automatic UV curve data collection and calculation.

Thus, the system disclosed herein includes one or more means to detect the contents of an eluant from the chromatography media. The detector may be a light-based detector which relies on multi-wavelength detection or single wavelength detection. Suitable detectors include a spectrophotometer capable of detecting visible wavelengths of light, a UV absorption detector, a fluorescence detector. The detector may be a light scattering detector which relies on a laser source or an electrochemical detector which responds to substances that are either oxidizable or reducible and the electrical output is an electron, flow generated by a reaction that takes place at the surface of the electrodes.

### III. Method for Informing Load Volumes

Disclosed herein are methods for informing load volumes for capture chromatography in system and methods for continuous manufacture of a biologic product, including but not limited to those described above.

The method disclosed herein provides a simple mathematical means to predict the average permeate concentration on a currently loading chromatography column (e.g., a ProA column) to inform how much volume of bioreactor permeate to load onto said column to be within a desired load challenge range. Advantageously, the method is cost effective and requires minimal development work to implement. The methods described herein are capable of holding load challenges within our acceptable ranges.

Specifically, a method is disclosed for controlling a current load challenge using the UV signal from previous elution cycles. Integrating the area under the elution curve and multiplying by a previously determined calibration constant to determine the mass of product eluted from the chromatography column. Dividing the elution mass by the volume loaded onto the column then gives a prediction of the average titer of the product during loading.

In one embodiment, an optionally computer implemented method for optimizing continuous chromatography (e.g., continuous protein A chromatography) in a system of continuous production of a biologic product (e.g. a perfusion bioreactor), comprising (i) taking an optical density (OD_measurement of the eluate from the protein A column; (ii) applying a mathematical formula to the OD measurement; and (iii) adjusting the continuous load of the protein A column to achieve the optimization.

The method utilizes a flow meter to measure the volume loaded onto the chromatography column and an optical absorbance detector, e.g., UV sensor to record the amount of ultraviolet or visible light absorbed by components of the mixture being eluted off the chromatography column. By passing UV light through the individual components of an eluting sample mixture and measuring the amount of UV light absorbed by each component. The purpose of the method is to control the load challenge onto the column within a target range.

Where the system is a continuous protein manufacturing system system described herein, no additional equipment is required to implement the method beyond what is already present in the design, i.e., the UV sensors in the column effluent for the purpose of general process control.

The flowmeter can be any suitable flow meter, e.g., a Levitronix flow meter.

The UV sensor can be any suitable UV sensor, fixed or variable wavelength, which includes diode array detector (DAD or PDA). The UV absorption of the effluent is continuously measured at single or multiple wavelengths. The wavelength may vary and in one embodiment, is between about 200 to about 400 nm or about 200 to about 800 nm. In certain embodiments, the wavelength is between about 190 nm to about 700 nm, more particularly 300 nm.

In certain embodiments, the method utilizes a flowmeter to measure the volume loaded onto a ProA affinity column and a UV sensor, known in the art such as an Optek probe, to record the optical density of the ProA elution profile at a wavelength of 300 nm. The A300 signal is scaled to match the intensity of an A280 signal.

The general principle is that the mass of protein that elutes from a Protein A column should be proportional to the mass loaded onto the column. The mass of product that elutes from the Protein A column can be determined by multiplying the volumetric flowrate by the optical density measured by post-column UV sensor, calculating the integral with respect to volume, and then evaluating the integral over the collected elution volume. Integration is performed by the trapezoidal rule. Dividing the elution mass by the volume loaded onto the column then gives a prediction of the average titer of the product during loading Advantageously, this method permits past titer values to be obtained without using an external instrument.

In one embodiment, the method comprises: (i) integrating the area under elution curve (OD*Liters) by (a) using the UV signal from 300 nm scaled to 280 nm and (b) approximating the integral using the trapezoid rule; (ii) converting UV area to elution mass (g) using molecule's mass extinction coefficient, εm; (iii) converting mass eluted to mass load using calibration parameter a (effectively yield); (iv) calculating average load titer (g/L) from mass loaded and volume loaded and (v) combing the equations corresponding to (i)-(iv) above.

The mathematic equation corresponding to the integrating in (i), above, is:

$${A}_{300} = Δ t ( {\sum }_{i = 0}^{n} \frac{{Q}_{i - 1} {D}_{i - 1} + {Q}_{i} {D}_{i}}{2} )$$

The mathematic equation corresponding to the converting in (ii), above is:

$${M}_{elute} = \frac{{A}_{300}}{{ε}_{m}}$$

The mathematic equation corresponding to the converting in (iii), above is:

$${M}_{load} = \frac{{M}_{elute}}{α}$$

The mathematic equation corresponding to the calculating in (iv), above is:

$${T}_{avg} = \frac{{M}_{load}}{{V}_{load}}$$

The full equation used to determine the product titer from using the elution UV method is as follows:

$$Titer = \frac{{k}_{col}}{2 {ε}_{m} {V}_{l}} ( {{\sum }^{n}}_{i = 0} {Q}_{i} {D}_{i} + {\sum }_{i = 0}^{n} {Q}_{i + 1} {D}_{i + 1} ) \qquad Equation 1$$

Titer (g/L)=average permeate titer while loading the column
V₁ (mL)=The volume of permeate loaded onto the column prior to elution
i=the number of timepoints that have elapsed since the start of elution collection
n=the number of timepoints that elapse between the start and end of collection of the eluate
Dᵢ=the i^{th} measurement of optical density at 300 nm by the Optek probe
Qᵢ=the i^{th} measurement of volumetric flowrate during elution. Q is calculated by summing the measured flow rate
s for the elution buffer concentrate and the elution buffer WFI diluent.
εₘ=The molecule specific extinction coefficient at 300 nm, with units of g/L/cm
k_{colA} and k_{colB}*=A constant determined by analysis of prior datasets, specific to each of the two Protein A columns in the continuous protein manufacturing system. This constant will be entered into the continuous protein manufacturing system's control system prior to the start of the batch.

It is necessary to use fitted constants for this calculation rather than applying Beer's law according to the molecule specific extinction coefficient for a number of reasons. First, although the UV sensors have been calibrated so that the A300 measurement should accurately reflect the A280 measurement, error in calibration have been observed in previous datasets. Using a fitted constant accounts for any persistent calibration offsets. Additionally, using a fitted constant allows the model to account for the fact that column yield is typically less than 100%. Each column should be calibrated separately because sensor calibration often varies between the two columns. Including separate calibration constants for each column improves the accuracy of the model prediction.

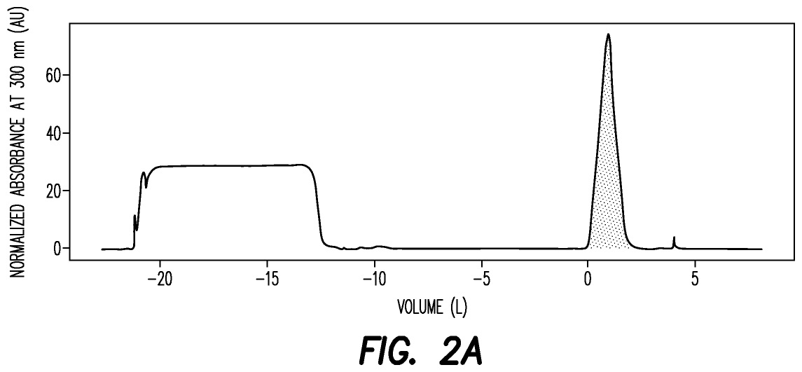

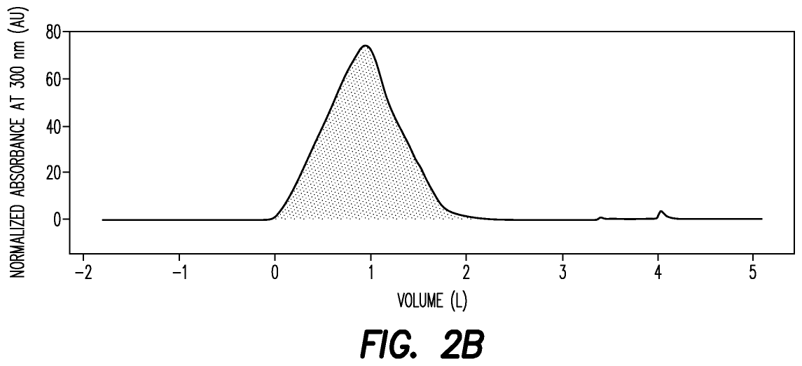

The values of the fitted constants are determined from historical data from eight separate batches run across three different sites with three different molecules. In a future implementation, the fitted constant will be determined by using a Protein A HPLC at the start of the first load cycle and comparing the mass eluted to the mass loaded. Timepoints were spaced every 10 seconds in the historical datasets. Protein A HPLC titer values were used as the calibration dataset. Elution masses from historical runs are calculated for each cycle by multiplying the volumetric flowrate by the optical density at 300 nm, summing this data over the elution collection (when A280 OD reached 0.20 and then collecting 3 CVs of eluate) and dividing the corresponding load volume for that cycle and the extinction coefficient of the mAb. FIG. 2 shows a chromatogram from a representative Protein A chromatography cycle, with the area under the curve of the elution represented as the shaded portion.

There are three methods for predicting the titer for the currently loading column using the titer from the previous elution, (i) the single-point method, (ii) the linear extrapolation method and (iii) a hybrid method. The single-point method risks (i) overchallenging the column when the titer is increasing very fast and (ii) overpredicting and underchallenging the column when the titer is decreasing. The linear extrapolation method works by fitting a line through the previous 3 titer measurements and using the line of the best fit to estimate the average titer of the next load cycle.

$${T}^{-} \_pred = m * t + b$$

m—Slope of the line of best fit for the last 3 measured titer values
t—Estimated time of next load midpoint
b—Y-intercept of best fit line for the last 3 measured titer values

When the actual titer is increasing, the linear extrapolation method is more likely to over predict the titer and thus underchallenge the column. Generally, overchallenging is worse than underchallenging the column. Risks of overchallenging the column include inaccurate titers from UV elution signal, potentially comprising product quality and/or sending more product down the drain.

The hybrid method uses a combination of the two approaches to provide an approach that is good for all titers: (i) positive slope from last three titers: linear extrapolation method and (ii) negative slope from last three titers: single point method.

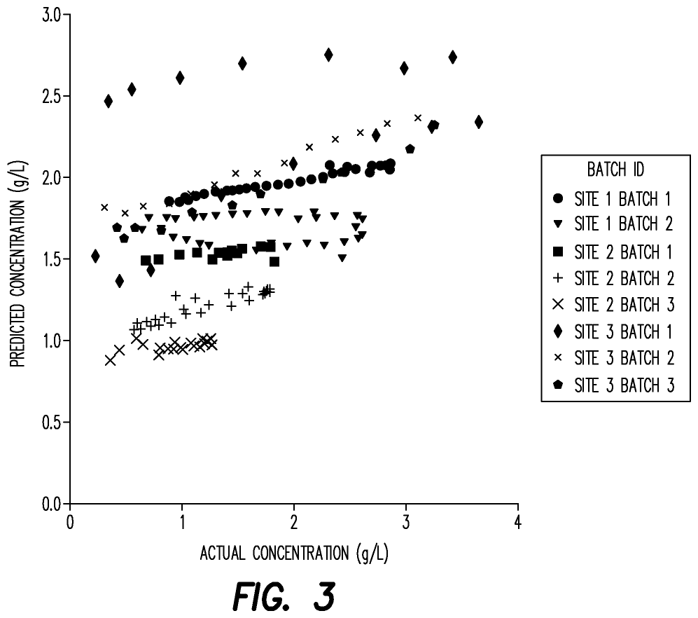

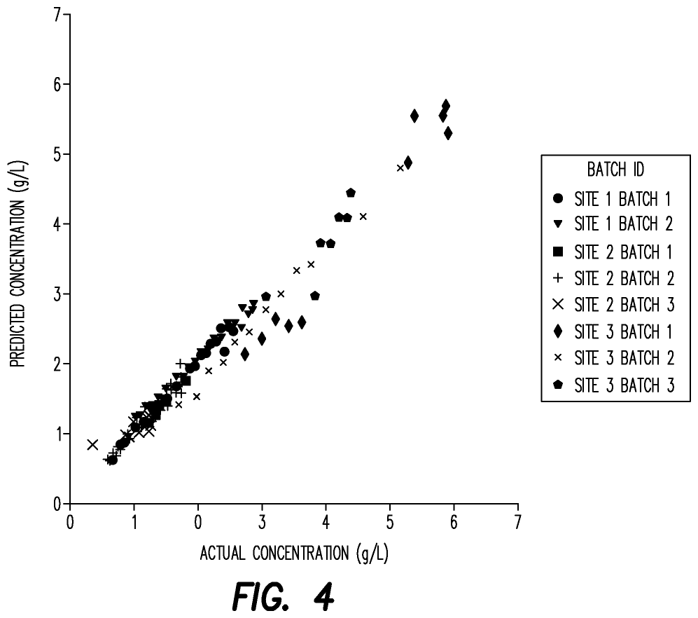

A linear regression was performed using the calculated elution masses as the predictor, and the average load titer as the response. The weighted average load titer is determined for each cycle by dividing the total mass of antibody loaded onto the column by the total volume of bioreactor permeate loaded onto the column. The intercept for this regression is forced to zero, and the regression slope is divided by the extinction coefficient for the calibration molecule to determine the calibration constant. The calibration constant needs to be determined once for each UV detector. This method was applied retroactively to eight batches performed with three separate molecules on three separate continuous protein manufacturing system systems. FIG. 3 shows the predicted vs. actual titers. The CV(RMSE) was determined to be 14.1%, indicating a high degree of accuracy. Within these datasets, many of the Protein A chromatography cycles had been overloaded, leading to product loss. This product loss impacted the size of the elution peak, introducing some error into the concentration predictions. Implementation of the titer prediction method would likely eliminate any instance of overloading. If these overloaded cycles are excluded from the dataset, the CV(RMSE) would improve to 10.5%. It is expected that this titer prediction method would be able to sufficiently control the Protein A load challenges such that overloading becomes a rare occurrence, and the expected variability of the system is likely closer to the 10.5% value obtained when excluding overloaded cycles from the dataset.

For runs where titer values change at fast rates, the elution UV titer prediction method may be less accurately, as the titer may change rapidly within the 3-7 hour lag between the mid-point of loading and elution of that cycle. The account for this, a linear-regression algorithm was developed to account for the rate of change of the titer. Linear regression was performed to determine the slope and y-intercept for the line of best fit for the three previous titer predictions. Linear regression was performed with the following equations:

$$m = \frac{n \sum {x}_{1} {y}_{i} - ( \sum {x}_{i} ) ( \sum {y}_{i} )}{n \sum {x}_{i}^{2} - {( \sum {x}_{i} )}^{2}} \qquad Equation 2$$

$$b = \frac{\sum {y}_{i} - m \sum {x}_{i}}{n}$$

m=slope
b=y-intercept
x=timepoint corresponding to the mid-point of the load that corresponds with the i^{th} titer prediction. The mid-point of loading was calculated by determining the total load volume for that cycle, and then establishing the time when half the total load volume had been applied to the column.
y=the i^{th} titer prediction from the elution UV method
n=the number of points in the regression

At each subsequent timepoint, the titer was estimated by multiplying the slope by the time elapsed since the mid-point of the previous load, and then adding the intercept. Choosing the time corresponding to the midpoint of loading as the x-value rather than the time of the elution allows the model to account for the potential change in titer between loading and elution. When only a single titer prediction has occurred in the batch, no linear regression is performed. Once three valid titer predictions have occurred we use linear regression to extrapolate the estimated titer for the next load cycle. To prevent potential instances of over-challenging the column when the linear regression slope is negative, or the titer is predicted to decrease, no linear regression is performed and instead uses the last titer measurement from the method to load the column.

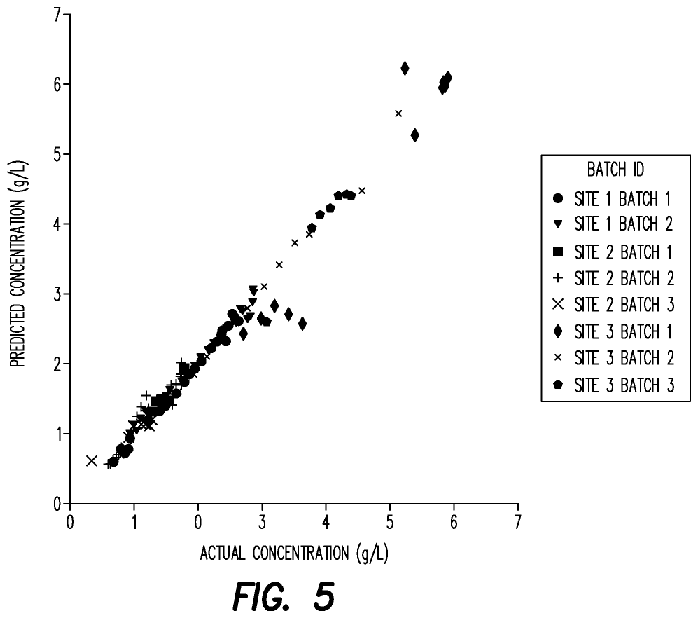

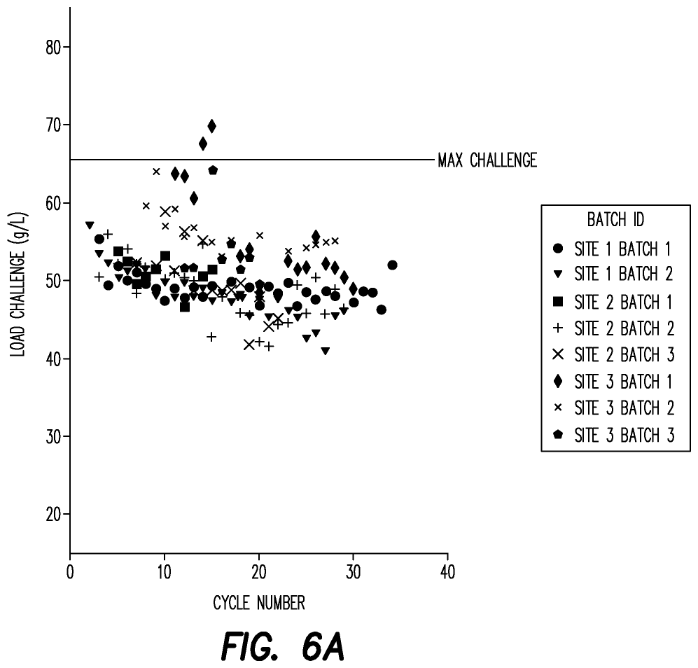

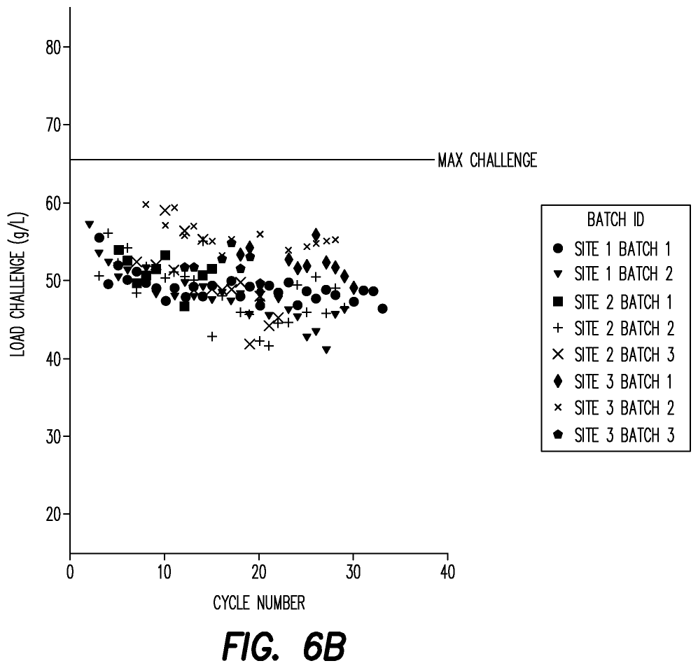

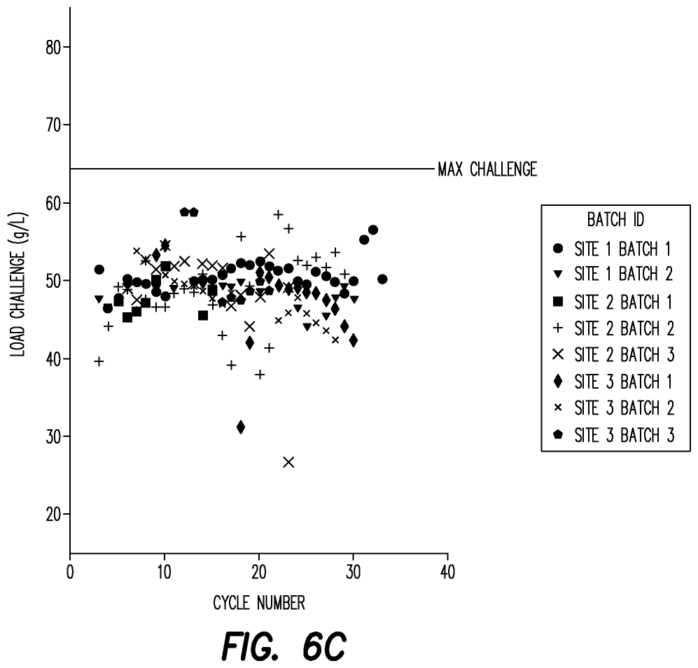

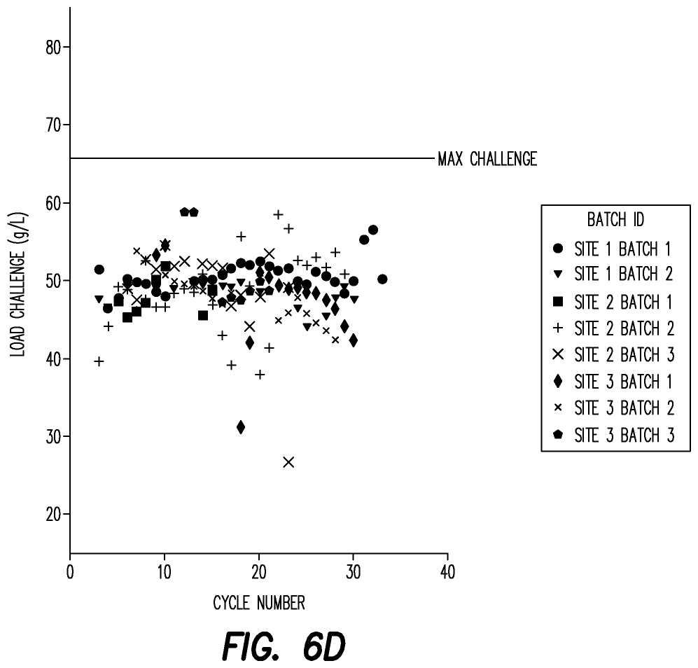

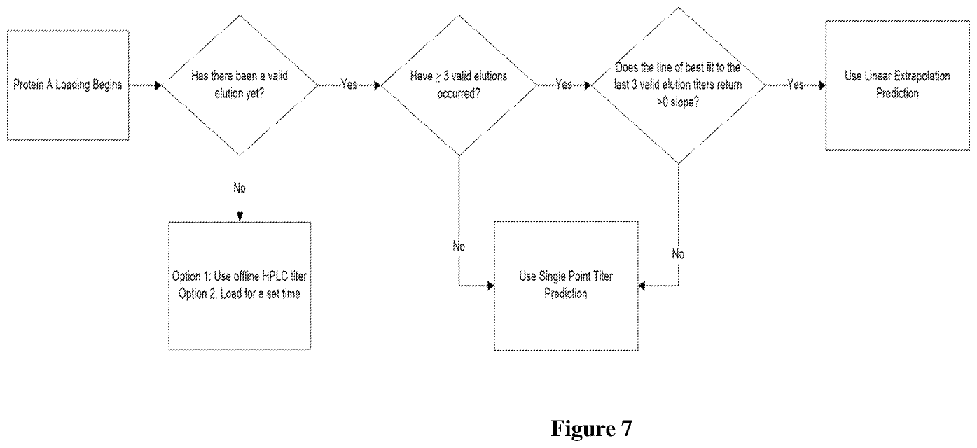

In one embodiment, the method permits the controlling the load challenge onto the Protein A column within a target load challenge range. Data from seven continuous protein manufacturing system batches was used to determine what the Protein A chromatography load challenges would have been if the elution UV titer prediction method had been employed with and without the linear regression adjustment. The target load challenge was assumed to be 50 g/L. FIG. 6 shows these results with and without the linear regression adjustment. Any cycle where a load time greater than 12 hours would be required to hit 50 g/L was omitted from the data set. It's clear that most runs are close to the 50 g/L target in all datasets. In the full dataset without linear regression (FIG. 5A), two runs would have had load challenges greater than 65 g/L and five runs would have had load challenges between 60 and 65 g/L. However, all these overloaded runs were the result of titer predictions based on overloaded runs. When a cycle is overloaded, the elution yield is lower than expected, which leads to an underprediction of the titer. When omitting runs where the titer prediction is based on an overloaded cycle (FIG. 6B), no runs would have been loaded over 65 g/L; in fact, all runs would have been below a 60 g/L challenge. Employing the elution UV titer method will enable robust control of Protein A loading, which will minimize the chances of overloading and subsequently increase the accuracy of the titer prediction. The method is further improved by employing the linear regression adjustment, as shown in FIG. 6C and FIG. 6D. When examining the full dataset with linear regression (FIG. 6C), only a single run would have had a load challenge >65 g/L, and only one additional run would have a load challenge between 60 and 65 g/L, significantly improving upon the results without linear regression. When predictions based on overloaded runs are omitted from the linear regression dataset, the prediction accuracy is improved to the point that no runs have a challenge greater than 60 g/L.

Advantageously, the method disclosed herein is not seriously impacted by (i) fast rate of change in titer; (ii) long pause in downstream operations (e.g., 24-48 hours); (iii) change in chromatography column DBC; or (iv) larger elution UV signal from impurities.

Overall, the elution UV titer prediction method is accurate enough to robustly control the protein A load challenge for most processes. For processes where the upstream titer is expected to rapidly change, the linear regression method can further improve the robustness of the prediction and reduce the likelihood of overloading. Prediction accuracy is calculated from the ratio of the predicted load titer versus the actual titer.

$$PredictionAccuracy ( \% ) = ( \frac{{\overline{C}}_{pred}}{{\overline{C}}_{HPLC}} )$$

The actual load titer (C⁻_HPLC) can be determined by interpolating titer measurements from permeate samples taken periodically throughout the day.

The method disclosed herein also has favorable yields. Process yield can be calculated by the ratio of mass eluted to mass loaded for the chromatography process:

$$Yield ( \% ) = ( \frac{{m}_{elu}}{{m}_{load}} )$$

The mass loaded (m_load) is calculated by numerical integration of the HPLC titer curve generated from periodic samples taken throughout the day and submitted to QC.

In one embodiment, a method is disclosed for capturing a biologic product from a feed stream containing the same, comprising (i) providing a feed stream, (ii) loading the feed stream onto at a chromatography column; and (iii) collecting the purified biologic product, wherein the feed stream has a load density within a target load density range or a specified load density. In one embodiment, the feed stream is loaded onto the chromatography column at about DBC of the chromatography materials for the biologic product.

In one embodiment, the target loading density range is between about 20 g/L and about 90 g/L, more particularly between about 20 g/L and about 80 g/L, about 30 g/L and about 70 g/L or about 40 and about 60 g/L.

In one embodiment, the target load density range is about between about 20 g/L and about 30 g/L, about 30 g/L and about 40 g/L, about 40 g/L and about 50 g/L, about 50 g/L and about 60 g/L and about 60 g/L and about 70 g/L, about 70 g/L and about 80 g/L, about 80 g/L and about 90 g/L or about 90 g/L and about 100 g/L.

In a particular embodiment, the specified load density is equal to or greater than about 20 g/L, about 25 g/L, about 30 g/L, about 35 g/L, about 40 g/L, about 45 g/L, about 50 g/L, about 55 g/L, about 60 g/L, about 65 g/L, about 70 g/L, about 75 g/L, about 80 g/L, about 85 g/L, about 90 g/L, or about 95 g/L or more.

In certain embodiments, the specified load density is less than about 50 g/L+/−2.

In a particular embodiment, the chromatography column is the first column within a multi-column continuous chromatography system.

In a particular embodiment, the chromatography column is an affinity chromatography column and more particularly, a ProA chromatography column.

In a particular embodiment, the chromatography column is the first chromatography column depicted in the system depicted in FIG. 1.

In certain embodiments, one or more parameters of the protein A matrix or resin (such as pH, ionic strength, temperature, the addition of other substances) is adjusted prior to contacting the protein A matrix or resin with a sample.

In certain embodiments, the feed stream include intact host cells and/or cellular debris. In certain embodiments, the feed stream is processed prior to loading onto the chromatography column, e.g., by filtration.

The flow rate may differ. In certain embodiments, the flow rate is between about 6 and about 20 CV/hr, more particularly about 6, about 8, about 10, about 12, about 14, about 16, about 18 or about 20 CV/hr or more.

The degree of purification obtained by the method disclosed herein may vary. In one embodiment, the target biologic product is purified by an amount greater than about 75%, about 80%, about 85%, about 90%, about 95%, or about 99%. In a particular embodiment, the target biologic product is purified by an amount between about 90 and about 99%, more particularly, about 92% and about 99%, about 94% and about 99%, about 96% and about 99% or about 98% and about 99%.

In certain embodiments, the productivity of the method disclosed herein is increased relative to a similar method that does not utilize the load optimization strategy disclosed herein. In a particular embodiment, the productivity of the method disclosed herein is increased by about 5%, about 10%, about 15%, about 20%, about 25%, about 30% or about 25% or more.

In certain embodiments, the facility footprint permitted by the method disclosed herein is decreased relative to a similar method that does utilize the load optimization strategy and in particular, utilizes a HPLC-based estimation strategy. In a particular embodiment, the facility footprint is reduced by about 1% to about 30%, more particularly, about 5% to about 20%, more particularly about 10%.

In certain embodiments, the eluate that results from the chromatography based purification that results from the method described herein is further processed and/or purified thereafter, e.g., further purified, inactivated, formulated or the like.

The overall goal of the methods and systems disclosed herein is the production of an isolated biologic product, e.g., an isolated protein or antibody (e.g., a monoclonal antibody).

The method disclosed herein is broadly applicable to any process producing a biologic product where a feed stream with a continually changing product concentration is loaded onto a bind-elute capture chromatography step. Additionally, the linear regression adjustment is broadly applicable to any bioprocess where any sort of process or quality attribute. Any suitable methods of capture chromatography could be employed, including ion-exchange, hydrophobic interaction, or mixed-mode chromatography. The only requirement is that chromatography cycle yield should be relatively consistent from run to run (as measured by actual yield v. predicted yield) in order to minimize the variability of the titer prediction.

The biologic product may be any biologic product having a UV-Vis absorbance in the range of 190 to 700 nm and in certain embodiments, is an monoclonal antibody.

As shown in FIG. 2 and FIG. 3, this method greatly improves upon the accuracy of the “feed absorbance minus effluent absorbance” method for determining product titer. When compared to the method of taking offline HPLC samples, this method requires much less labor as it can be implemented in a fully automated fashion through the distributed control system. Compared against the method of using an in-line HPLC with automated sampling, this method requires less equipment, requires less equipment maintenance, and requires no sampling of the bioreactor permeate, reducing the risk of contamination. Compared against the method of using Raman or FTIR spectroscopy, this method is easier to calibrate and requires less expensive equipment. Compared to methods that control protein A loading by always loading until product breakthrough is observed (insert citation here), this proposed method does not require loading of the Protein A column until product loss occurs, which would result in improved process yields compared to loading until breakthrough. Additionally, the breakthrough method relies on UV absorbance measurements taken from the bioreactor permeate stream, which contains many impurities that can potentially foul the UV sensor. This proposed method relies on UV measurements taken from the protein A elution, which is typically contains far lower levels of impurities than the bioreactor permeate stream, increasing the likelihood that an accurate UV absorbance measurement can be obtained.

### IV. Compositions

Disclosed herein are biologic products produced by the systems and methods disclosed herein.

In one embodiment, the biologic product is any biologic product having a UV-Vis absorbance in the range of 190 to 700 nm.

In one embodiment, the biologic product is a polypeptide, protein or antibody (e.g., a monoclonal antibody) and in particular, a polypeptide, protein or antibody for administration to a subject (e.g. a human). In certain embodiments, the antibody is a monoclonal antibody. In certain embodiments, the biologic product is a binding fragment.

In certain embodiments, the biologic product incorporated into pharmaceutical compositions suitable for administration to a subject. Typically, the pharmaceutical composition comprises an antibody and a pharmaceutically acceptable carrier. Pharmaceutical compositions comprising biologic product(s) purified using the systems and methods disclosed herein may assume a variety of form, i.e., dosage forms.

Methods of manufacturing or producing of the biologic product of interest known in the art may be used in combination with the systems and methods described herein. For example, a person of skill in the art knows how to manufacture or produce biologic products, such as recombinant proteins, using fermentation. In certain embodiments, the production of biologic product of interest comprises cultivating the eukaryotic cell expressing the biologic product of interest in cell culture. Cultivating the eukaryotic cell expressing the biologic product of interest in cell culture may comprise maintaining the eukaryotic cells in a suitable medium and under conditions that allow growth and/or protein production/expression. The biologic product of interest may be produced by fed-batch or continuous cell culture. Thus, the eukaryotic cells may be cultivated in a fed-batch or continuous cell culture, preferably in a continuous cell culture.

In certain embodiments, the eukaryotic host cells are yeast cells. In one embodiment, the eukaryotic host cell is a mammalian cell. Mammalian cells as used herein are mammalian cells lines suitable for the production of a secreted recombinant therapeutic protein and may hence also be referred to as “host cells”. In certain embodiments, the mammalian cells are rodent cells such as hamster cells. The mammalian cells are isolated cells or cell lines. In certain embodiments, the mammalian cells are transformed and/or immortalized cell lines. In certain embodiments, the mammalian cells are adapted to serial passages in cell culture and do not include primary non-transformed cells or cells that are part of an organ structure. In certain embodiments, the mammalian cells are BHK21, BHK TK-, Jurkat cells, 293 cells, HeLa cells, CV-1 cells, 3T3 cells, CHO, CHO-K1, CHO-DXB11 (also referred to as CHO-DUKX or DuxB11), a CHO-S cell and CHO-DG44 cells or the derivatives/progenies of any of such cell line. In certain embodiments, the mammalian cells are CHO cells, such as CHO-DG44, CHO-K1 and BHK21, and even more preferred are CHO-DG44 and CHO-K1 cells. In certain embodiments, the mammalian cells are CHO-DG44 cells. Glutamine synthetase (GS)-deficient derivatives of the mammalian cell, particularly of the CHO-DG44 and CHO-K1 cell are also encompassed. In one embodiment, the mammalian cell is a Chinese hamster ovary (CHO) cell, for example a CHO-DG44 cell, a CHO-K1 cell, a CHO DXB11 cell, a CHO-S cell, a CHO GS deficient cell or a derivative thereof.

In certain embodiments, the host cell may further comprise one or more expression cassette(s) encoding a heterologous protein, such as a therapeutic protein, for example a recombinant secreted therapeutic protein. In certain embodiments, the host cells may also be murine cells such as murine myeloma cells, such as NSO and Sp2/0 cells or the derivatives/progenies of any of such cell line.

The expression of the biologic product of interest or recombinant protein occurs in a cell comprising a DNA sequence coding for the biologic product of interest or recombinant protein, which is transcribed and translated into the protein sequence including post-translational modifications to produce the biologic product of interest or recombination protein in cell culture.

Disclosed herein is a method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) capturing or purifying the biologic product of interest comprising continuous chromatography of the fluid feed comprising biologic product of interest and one or more impurities or buffer components; and
(IV) optionally formulating the biologic product of interest into a pharmaceutically acceptable formulation suitable for administration; and
wherein the method further comprises:
(i) taking an optical density (OD) measurement of an eluate from a chromatography column; (ii) applying a mathematical formula to the OD measurement, wherein the mathematical formula is Equation 1; and (iii) adjusting the continuous load of the chromatography column to achieve the optimization.

Disclosed herein is a method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) capturing or purifying the biologic product of interest comprising continuous chromatography of the fluid feed comprising biologic product of interest and one or more impurities or buffer components; and
(IV) optionally formulating the biologic product of interest into a pharmaceutically acceptable formulation suitable for administration; and
wherein the method further comprises:
(i) taking an optical density (OD) measurement of an eluate from a chromatography column; (ii) applying a mathematical formula to the OD measurement, wherein the mathematical formula is Equation 2; and (iii) adjusting the continuous load of the chromatography column to achieve the optimization.

In certain embodiments, the biologic product of interest is a recombinant protein. In certain embodiments, the step of cultivating a eukaryotic cell expressing the biologic product of interest in cell culture occurs in a fed-batch cell culture. In certain embodiments, wherein the step of cultivating a eukaryotic cell expressing the biologic product of interest in cell culture occurs in a continuous cell culture.

The following examples are presented for illustrative purposes only and are not intended to be limiting.

### EXAMPLES

### Example 1: Analytical Methods

Online Pro A titer chromatography was performed using a Waters (Milford, MA) PATROL UPLC Process Analysis System sampling through an Artesyn valve directly from the permeate line. Chromatographic separation was performed by injecting 5 μL from a fixed sample loop on a POROS A 20 μm, 2.1×30 mm column (Thermo Fisher Scientific, Waltham, MA) with a 0.5 μm precolumn filter cartridge (IDEX, Lake Forest, IL). Mobile phase A is phosphate buffered saline at pH 7.4 and mobile phase B is phosphate buffered saline at pH 2.2. Sample is loaded at either 80% mobile phase A and 20% mobile phase B or 100% mobile phase A. Elution is performed via gradient elution, with a final buffer ratio of 100% mobile phase B. Detection was by absorbance at 280 nm. On-line samples are quantitated against a standard curve.

### Example 2. Calculation of Permeate Titer in the Continuous Protein Manufacturing System Through Integration of the Elution UV Signal from the Protein a Chromatography

This example describes the procedure for calculating the permeate titer in the Continuous Protein Manufacturing System through integration of the elution UV signal from the Protein A chromatography step. The general principle is that the mass of protein that elutes from a Protein A column should be proportional to the mass loaded onto the column. The mass of product that elutes from the Protein A column can be determined by integrating the area under the curve for the UV signal from the post-column UV sensor during elution. Dividing the elution mass by the volume loaded onto the column then gives a prediction of the average titer of the product during loading.

The full equation used to determine the product titer from using the elution UV method is as follows:

$$Titer = {k}_{col} \times \frac{Δ t}{{V}_{load}} \times \frac{1}{n} {\sum }_{i}^{n} {OD}_{i} \qquad Equation 3$$

Titer (g/L)=average permeate titer while loading the column
V_{load} (mL)=The volume of permeate loaded onto the column prior to elution
ODᵢ=the i^{th} measurement of optical density at 300 nm by the Optek probe
Δt (s)=The total elapsed time during the elution when product is being collected
k_{colA} and k_{colB}*=A constant determined by analysis of prior datasets, specific to each of the two Protein A columns in the continuous protein manufacturing system. This constant will be entered into DeltaV prior to the start of the batch.
n=the number of timepoints that elapse between the start and end of collection of the eluate
   * It is necessary to use fitted constants for this calculation rather than applying Beer's law according to the molecule specific extinction coefficient for a number of reasons. First, although the UV sensors have been calibrated so that the A300 measurement should accurately reflect the A280 measurement, error in calibration have been observed in previous datasets. Using a fitted constant accounts for any persistent calibration offsets. Additionally, using a fitted constant allows the model to account for the fact that column yield is typically less than 100%. Each column should be calibrated separately because sensor calibration often varies between the two columns. Including separate calibration constants for each column improves the accuracy of the model prediction.

### Operation Sequence:

a. Prior to the run
   i. k_{colA} and k_{colB} values will be established by Pfizer/BI through analysis of previous data or from the first elution cycle with the use of offline Protein A HPLC data.
   ii. k_{colA} and k_{colB} values will be programmed into the run recipe.
b. During the run
   i. Before the system begins loading of the Protein A column, an initial titer value must be manually entered into DeltaV based on offline Protein A HPLC data.
   ii. Once the first Protein A column elution occurs, DeltaV should calculate the titer based on Equation 1, and then update the current titer in DeltaV with the model-based titer.
   iii. With every Protein A column elution, a new titer should be calculated and then used to update the current titer.
   iv. The user should have the ability to manually overwrite the titer as necessary by manually entering a titer value into DeltaV
   v. If any errors occur that prevent DeltaV from calculating an accurate titer based on the UV model, a warning should be displayed to alert the user to the issue.

### DeltaV Requirements

c. Create a variable that stores the current titer prediction
   i. The current titer value should be able to be updated manually by user entry or updated automatically by DeltaV using the elution UV algorithm
   ii. There should be an option to turn off automatic updating of the titer from the elution UV method
d. Accept two values for constants, k_{colA} and k_{colB}, which will be manually entered into DeltaV prior to the start of the batch.
e. Record the volume of permeate loaded onto each Protein A column per cycle
   i. The permeate loading totalizer should start when a column begins loading, and end as soon as loading switches to the other column or buffer begins flowing over the original column.
   ii. When the system switches to begin loading the other column or flowing buffer over the original column, the final value of the flow totalizer should be recorded into a new variable which stores the previous load volume and the flow totalizer should reset
   iii. If permeate flow is sent to waste, the flow totalizer should stop counting flow but should not reset or overwrite the previous load volume variable
f. Record the duration (in time) of the collection of the Protein A eluate
g. Record absorbance values at 300 nm during elution collection
   i. The interval between absorbance value measurements should not be more than 10 seconds
   ii. The time interval between absorbance value measurements should be consistent across the batch
h. Determine the average absorbance at 300 nm across all timepoints obtained during collection of the Protein A eluate
i. As soon as elution collection has finished, apply Equation 1 to calculate the titer prediction
   i. Equation 1 should be calculated using k_{colA} when using elution data from column A, and k_{colB} when using elution data from column B
j. Update the titer variable with the prediction from Equation 1
k. Use the value of the titer variable to control the loading duration for the two Protein A columns
   i. As soon as the titer variable value is overwritten, either manually or with another prediction from the elution UV model, use the new titer value to calculate loading duration
l. After completion of the run, the user should be able to access the titer prediction data. For each cycle, the user should be able to access the following data:
   i. Column load volume
   ii. Elution duration
   iii. Titer prediction

### Error Handling

m. If an elution occurs but the elution UV model cannot be successfully applied for any reason, do not overwrite the previous titer prediction and provide a warning in DeltaV that the prediction was unsuccessful
n. If the elution collection duration is less than 12 minutes, do not overwrite the previous titer prediction and provide a warning to the user that the titer model prediction could not accurately predict the titer because the elution collection duration was shorter than expected
o. If the elution collection volume is determined to be greater than 4.0 Protein A column volumes (CVs), do not overwrite the previous titer prediction and provide a warning to the user that the titer model prediction could not accurately predict the titer because the elution collection volume was greater than expected
   i. The elution collection volume in CVs should be determined by multiplying the average elution flowrate by the duration of the elution, and then dividing by the volume of the Protein A column
p. If the A300 signal at any point during elution collection exceeds the maximum A300 value of the Optek probe, do not overwrite the previous titer prediction and a warning should be displayed alerting the user that the detector has been saturated.

### Example 3: Comparative Example

International Publication No. WO2010151214A1 describes a spectroscopic method in which UV sensors were placed in the feed and effluent streams of a Protein A capture chromatography system. Protein A chromatography is highly selective for monoclonal antibody. During loading of the column, mAb present in the feed will bind to the column, but impurities will flow through the column. As such, the effluent stream will essentially have the same composition as the feed stream, minus the presence of antibody. UV sensors placed in the column feed and column effluent measure the total absorbance at 280 nm. By subtracting the absorbance in the effluent from the absorbance in the feed and dividing by the molecule specific extinction coefficient, the antibody titer can be determined. There are however some practical difficulties in using this method. Absorbance measurements in complex streams tend to have relatively high noise, reducing the accuracy of any titer prediction based on this measurement. Additionally, UV sensors are prone to fouling when exposed to complex feed-streams for long periods of time. To illustrate, this method was applied to try to predict the titer across eight separate continuous batches, and the results are shown in FIG. 2. Linear regression was used to determine the relationship between the difference in absorbance between the feed and effluent streams and the actual concentration, as measured by Protein A HPLC. To create a fully continuous time series of Protein A HPLC concentrations for model calibration, linear interpolation was used to estimate the antibody concentration between Protein A HPLC measurements. Each batch was treated as a separate categorical variable in the linear regression. Model error was measured by calculating the coefficient of variation of the root-mean-square error (CV(RMSE)). The CV(RMSE) was determined to be 45%, indicating a relatively poor predictive capacity when this method is applied to real-world data.

According to one embodiment, the methods herein are implemented by one or more computing devices. The optionally computer implemented program product may include a computer readable storage medium (or media) having computer readable program instructions thereon for causing a processor to carry out aspects of the method disclosed herein.

In certain embodiments, a warning is incorporate into code when the elution mass is greater than a threshold setting (e.g., ˜60 g/L). That elution titer could be flagged and not considered “valid”. If a non-valid titer is used in a prediction, the code could target a lower load challenge until all three previous titers are “valid elutions.”

In the preceding specification, various embodiments have been described with reference to the examples. It will, however, be evident that various modifications and changes may be made thereto, and additional embodiments may be implemented, without departing from the broader scope of the exemplary embodiments as set forth in the claims that follow. The specification and drawings are accordingly to be regarded in an illustrative rather than restrictive sense.

1-23. (canceled)

24. A method for optimizing continuous chromatography in a system of continuous production of a biologic product, comprising (i) taking an optical density (OD) measurement of an eluate from a chromatography column; (ii) applying a mathematical formula to the OD measurement, wherein the mathematical formula is Equation 1:
$$Titer = \frac{{k}_{col}}{2 {ε}_{m} \text{?}} ( {{\sum }^{n}}_{i = 0} {Q}_{i} \text{?} + {\sum }_{i = 0}^{n} {Q}_{i + 1} {D}_{i + 1} )$$
$$\text{?} \text{indicates text missing or illegible when filed}$$
and (iii) adjusting the continuous load volume of the chromatography column to achieve the optimization.

25. The method of claim 24, wherein the biologic product is a protein.

26. The method of claim 24, wherein the biologic product is a monoclonal antibody.

27. A method for optimizing continuous chromatography in a system of continuous production of a biologic product, comprising (i) taking an optical density (OD) measurement of an eluate from a chromatography column; (ii) applying a mathematical formula to the OD measurement, wherein the mathematical formula is Equation 2:
$$m = \frac{n \sum {x}_{1} {y}_{i} - ( \sum {x}_{i} ) ( \sum {y}_{i} )}{n \sum {x}_{i}^{2} - {( \sum {x}_{i} )}^{2}}$$
$$b = \frac{\sum {y}_{i} - m \sum {x}_{i}}{n} ;$$
and (iii) adjusting the continuous load of the chromatography column to achieve the optimization.

28. The method of claim 27, wherein the biologic product is a protein.

29. The method of claim 27, wherein the biologic product is a monoclonal antibody.

30. The method of claim 24, wherein the method does not utilize high performance liquid chromatography.

31. The method of claim 24, wherein the optimization comprises maintaining load density to remain within a predetermined, target load density or target load density range.

32. The method of claim 31, wherein the predetermined, target load density is less than about 100 g/L.

33. The method of claim 31, wherein the predetermined, target load density is less than about 65 g/L.

34. The method of claim 31, wherein the system permits processing up to 6 g/L/day of the biologic product.

35. A method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) capturing or purifying the biologic product of interest, wherein said capturing or purifying comprises continuous chromatography of the fluid feed comprising biologic product of interest and one or more impurities or buffer components; and
(IV) optionally formulating the biologic product of interest into a pharmaceutically acceptable formulation suitable for administration; and
wherein the method further comprises:
(i) taking an optical density (OD) measurement of an eluate from a chromatography column; (ii) applying a mathematical formula to the OD measurement, wherein the mathematical formula is Equation 1:
$$Titer = \frac{{k}_{col}}{2 {ε}_{m} \text{?}} ( {{\sum }^{n}}_{i = 0} {Q}_{i} \text{?} + {\sum }_{i = 0}^{n} \text{?} {D}_{i + 1} ) ;$$
$$\text{?} \text{indicates text missing or illegible when filed}$$
and (iii) adjusting the continuous load of the chromatography column to achieve the optimization.

36. The method of claim 35, wherein the biologic product of interest is a recombinant protein.

37. The method of claim 35, wherein the step of cultivating a eukaryotic cell expressing the biologic product of interest in cell culture occurs in a fed-batch cell culture.

38. The method of claim 35, wherein the step of cultivating a eukaryotic cell expressing the biologic product of interest in cell culture occurs in a continuous cell culture.

39. A method of manufacturing a biologic product of interest comprising the steps of:
(I) cultivating a eukaryotic cell expressing the biologic product of interest in cell culture;
(II) harvesting the biologic product of interest from the cell culture in the form of a fluid feed comprising biologic product of interest and one or more impurities or buffer components;
(III) capturing or purifying the biologic product of interest, wherein said capturing or purifying comprises continuous chromatography of the fluid feed comprising biologic product of interest and one or more impurities or buffer components; and
(IV) optionally formulating the biologic product of interest into a pharmaceutically acceptable formulation suitable for administration; and
wherein the method further comprises:
(i) taking an optical density (OD) measurement of an eluate from a chromatography column; (ii) applying a mathematical formula to the OD measurement, wherein the mathematical formula is Equation 2:
$$m = \frac{n \sum {x}_{1} {y}_{i} - ( \sum {x}_{i} ) ( \sum {y}_{i} )}{n \sum {x}_{i}^{2} - {( \sum {x}_{i} )}^{2}}$$
$$b = \frac{\sum {y}_{i} - m \sum {x}_{i}}{n} ;$$
and (iii) adjusting the continuous load of the chromatography column to achieve the optimization.

40. The method of claim 39, wherein the biologic product of interest is a recombinant protein.

41. The method of claim 39, wherein the step of cultivating a eukaryotic cell expressing the biologic product of interest in cell culture occurs in a fed-batch cell culture.

42. The method of claim 39, wherein the step of cultivating a eukaryotic cell expressing the biologic product of interest in cell culture occurs in a continuous cell culture.

43. The method of claim 24, wherein said method is computer implemented.

44. The method of claim 27, wherein the method is computer implemented.

45. The method of claim 35, wherein the method is computer implemented.

46. The method of claim 39, wherein the method is computer implemented.
